(19) United States
(12) Patent Application Publication
(10) Pub. No.: US 2024/0123180 A1
(43) Pub. Date: Apr. 18, 2024

(54) Wristwatch System for Detecting Negative Emotional State of a Wearer

(71) Applicant: Sweetie Stewart, Windermere, FL (US)

(72) Inventor: Sweetie Stewart, Windermere, FL (US)

(21) Appl. No.: 18/149,702

(22) Filed: Jan. 4, 2023

Related U.S. Application Data
(60) Provisional application No. 63/417,121, filed on Oct. 18, 2022.

(51) Int. Cl. A61M 21/02

(52) U.S. Cl. CPC A61M 21/02; A61M 2021/0027; A61M 2230/04; A61M 2230/65

(57) ABSTRACT

The present invention relates to a wearable smartwatch system for detecting negative emotions of a wearer of the smartwatch of the system. The system includes an earbud coupled to the smartwatch using a Bluetooth connection. More specifically, the smartwatch includes a plurality of electrodes for measuring HRV and electrodermal activity of the wearer. A processor of the smartwatch processes the data measured by the electrodes to determine emotional state of the wearer. A pre-recorded audio is automatically played out through the earbud to help calm down the wearer upon determination of the negative emotional state. In some embodiments, an alert signal is sent to a parent, teacher, or other caregiver to obtain assistance for a wearer to cope with negative emotions and feelings.

### CROSS-REFERENCE TO RELATED APPLICATION

The present application claims priority to, and the benefit of, U.S. Provisional Application No. 63/417,121, which was filed on Oct. 18, 2022, and is incorporated herein by reference in its entirety.

### FIELD OF THE INVENTION

The present invention relates generally to the field of smartwatches. More specifically, the present invention relates to a novel smartwatch system for detecting negative emotions such as stress and anxiety of a wearer of the watch. The system has an earbud coupled with the smartwatch for playing out pre-recorded messages to help the wearer coping with emotions. An alert signal is also transmitted by the smartwatch to a wireless electronic device of a parent, teacher, or other caregiver to obtain assistance in coping with emotions and feelings of the wearer of the device. Accordingly, the present disclosure makes specific reference thereto. Nonetheless, it is to be appreciated that aspects of the present invention are also equally applicable to other such as applications, devices, and methods of manufacture.

### BACKGROUND

By way of background, mental health and emotional state are of prime importance. Good emotional health keeps a person in good energy, positive outlook, helps in maintaining a clear focus and thinking. Individuals who are emotionally healthy are in control of their thoughts, feelings, and behaviors, and are able to cope with all the challenges. However, due to irregular routines, unhealthy lifestyles, peer pressure, and lack of social connect, individuals have common troubles dealing with negative emotions such as stress, frustration, and anxiety. This often leads to poor emotional health that may deplete mental resources and leads to exhaustion. Poor emotional health leads to challenges, such as becoming upset and stressed easily, and in extreme conditions may lead to depression and mental diseases.

Commonly, caregivers, family members, and friends help individuals in coping with negative and poor emotional state. Further, many times, family and friends are late or delayed in becoming aware about the mental state of their loved ones which not only reduces the pace of healing but adversely affects the person suffering from negative thoughts. In some cases, a person may take medicines and therapies which is not only expensive but are also discomforting and time consuming.

Individuals generally see changes in their behavior to know about negative emotions and therefore, it is desired to have a device and method that automatically detects a negative emotional state of a user. Further, caregivers, family members and friends desire a device that can alert them quickly about negative state of their loved ones.

Many students find it challenging to focus for a long time and need instructions from teachers and parents. Lack of focus may affect their performance and therefore, both students and teachers need a device that helps in improving focus and motivation in order for students to study for longer periods.

Therefore, there exists a long felt need in the art for a device that helps individuals in coping with their emotions and feelings. There is also a long felt need in the art for a device that automatically detects the negative emotional state of a person. Additionally, there is a long felt need in the art for a device that helps in reducing the effect of negative emotions on a person. Moreover, there is a long felt need in the art for a device that helps students to improve focus for a long time. Further, there is a long felt need in the art for a device that reduces dependency on medicines and therapies for reducing and coping with negative emotions and states. Furthermore, there is a long felt need in the art for a device that identifies mood changes of a user through biological effects such as changes in breathing, high blood pressure, and more. Finally, there is a long felt need in the art for an improved device that is easy to use, is non-invasive and helps the user and individuals therearound in coping with the effects of negative emotions.

The subject matter disclosed and claimed herein, in one embodiment thereof, comprises a novel smartwatch system configured to reduce the effect of negative emotions of a wearer of the smartwatch. The system includes a smartwatch and an earbud, the smartwatch is designed to wrap around a wrist of the wearer and can be wirelessly coupled to the earbud using a wireless connection. The smartwatch has a plurality of electrodes for measuring bio signals of the wearer and a processor for determining the emotional state of the wearer using the measured bio signals by the electrodes. An internal memory of the smartwatch stores a plurality of prerecorded encouraging and motivating messages that are automatically played out by the earbud when the processor determines emotional state of the wearer is negative and stressful, thus helping in reducing effect of negative thoughts. The smartwatch has a touch display for receiving wearer instructions and displaying vital signs and emotional state of the wearer. A help button is configured to be pressed by the wearer to send an alert signal or message to a remote wireless device to obtain assistance in coping with emotions and feelings.

In this manner, the smartwatch system of the present invention accomplishes all of the forgoing objectives and provides users with an improved wristwatch capable of reducing the effect of negative emotions. The watch detects negative emotions such as stress and anxiety, sounding an alert or other pre-recorded audio through the earbud and helping calm down the wearer. The watch ensures that the wearer can identify and cope with emotions and identify mood changes through biological effects such as changes in breathing, high blood pressure, and more. Parent, teacher, or other caregiver can also be alerted to obtain assistance in coping with their emotions and feelings.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a smartwatch system configured to reduce the effect of negative emotions of a wearer of the smartwatch. The system includes a smartwatch and an earbud, the smartwatch is designed to wrap around a wrist of the wearer and wirelessly couple to the earbud using a Bluetooth connection. The smartwatch has a plurality of electrodes for measuring heart rate variability, blood pressure, and electrodermal activity of the wearer and a processor for determining the emotional state of the wearer using the measured vital signs by the electrodes. An internal memory of the smartwatch stores a plurality of prerecorded encouraging and motivating messages that are automatically played out by the earbud when the processor determined emotional state of the wearer is negative and stressful. The smartwatch has a display for displaying vital signs and emotional state of the wearer and has a help button which is pressed by the wearer to send an alert signal or message to a remote wireless device to obtain assistance in coping with emotions and feelings. The processor has machine learning intelligence to automatically detect emotional state of the wearer based on the values detected by the electrodes.

In yet another embodiment, a wearable device system for detecting and improving emotional state of a user is disclosed. The wearable device system comprising a smartwatch and a paired earbud, the smartwatch including a touch display for receiving input from the user and displaying vital signs and emotional state of the user wherein the user is wearing the smartwatch; one or more electrodes for detecting one or more bio signals of the user; a processor for processing said detected one or more bio signals to detect an emotional state of the user; an internal memory for storing encouraging and motivational audio messages wherein at least one audio message is transmitted to the earbud for playing out upon detecting a negative emotional state of the user; the audio message helps the user coping the negative emotional state; a help button disposed in the housing of the smartwatch configured to be pressed by the user wherein an alert signal is transmitted to a remote wireless device to obtain assistance in coping with emotions and feelings. The smartwatch can also be connected to a cloud server for storing the vital signs and emotional state information of the user for secure storage and for receiving machine learning classifiers based on profile of the user for accurately detecting the emotional state of the user.

In yet another embodiment, the smartwatch has a location unit for detecting current location of the watch and a wireless transceiver for establishing a wireless channel with the earbud. The alert signal transmitted to the remote wireless device includes the current location of the watch detected by the location unit.

In yet another embodiment, the smartwatch includes a battery for providing electrical power to the electrodes, processor, and display device. Further, the smartwatch stores a plurality of pre-recorded motivational and soothing messages.

In yet another embodiment, a method for reducing the effect of negative emotions upon a user is described. The method includes the steps of providing a smartwatch and an earbud; the smartwatch includes a plurality of electrodes for detecting vital signs of the user, a processor for processing the detected vital signs to determine an emotional state of the user, a memory for storing counterthoughts, emotions, and encouraging messages, a wireless transceiver for connecting to the earbud; measuring bio signals and vital signs of the user utilizing the electrodes; determining and compiling an emotional state of the user utilizing the processor; upon determining a negative emotional state of the user, communicating at least one message from the memory to the earbud; playing out the received message by the earbud; receiving actuation of a help button disposed on the smartwatch; and, transmitting an alert signal indicating an emotional state of the user to obtain assistance in coping with emotions and feelings.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an emotional imbalance detecting smartwatch system of the present invention in accordance with the disclosed architecture;

FIG. 2 illustrates a rear perspective view of the housing showing internal components of the watch in accordance with the disclosed architecture;

FIG. 3 illustrates a schematic view showing the wireless connection of the smartwatch and the earbud in accordance with the disclosed architecture;

FIG. 4 illustrates a perspective view showing a child using the multipurpose stress relieving watch system for coping with emotions, anxiety, and stress in accordance with the disclosed architecture;

FIG. 5 illustrates a flow diagram showing the process of determining stress and emotional state of a wearer of the watch in accordance with the disclosed architecture;

FIG. 6 illustrates a schematic view showing the connection of the watch with a remote wireless device and a cloud server in accordance with the disclosed architecture;

FIG. 7 illustrates a flow diagram depicting a process of associating a user/wearer with the system of the present invention in accordance with the disclosed architecture; and

FIG. 8 illustrates a flow diagram depicting a process of measuring and displaying the vital signs and emotional state of a wearer of the smartwatch in accordance with the disclosed architecture.

### DETAILED DESCRIPTION OF THE PRESENT INVENTION

The innovation is now described with reference to the drawings, wherein such as reference numerals are used to refer to such as elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a device that helps individuals in coping with their emotions and feelings. There is also a long felt need in the art for a device that automatically detects the negative emotional state of a person. Additionally, there is a long felt need in the art for a device that helps in reducing the effect of negative emotions on a person. Moreover, there is a long felt need in the art for a device that helps students to improve focus for a longer period time. Further, there is a long felt need in the art for a device that reduces dependency on medicines and therapies for reducing and coping with negative emotions and states. Furthermore, there is a long felt need in the art for a device that identifies mood changes of a user through biological effects such as changes in breathing, high blood pressure, and more. Finally, there is a long felt need in the art for an improved device that is easy to use, is non-invasive, and assists the user and individuals therearound in coping with the effects of negative emotions.

The present invention, in one exemplary embodiment, is a method for reducing the effect of negative emotions upon a user is described. The method includes the steps of providing a smartwatch and an earbud; the smartwatch includes a plurality of electrodes or sensors for measuring vital signs of the user, a processor for processing the detected vital signs to determine an emotional state of the user, a memory for storing encouraging messages, a wireless transceiver for connecting to the earbud; measuring bio signals and vital signs of the user utilizing the electrodes; determining and compiling an emotional state of the user utilizing the processor; upon determining a negative emotional state of the user, communicating at least one message from the memory to the earbud; playing out the received message by the earbud; receiving actuation of a help button disposed on the smartwatch; and, transmitting an alert signal indicating an emotional state of the user to obtain assistance in coping with emotions and feelings.

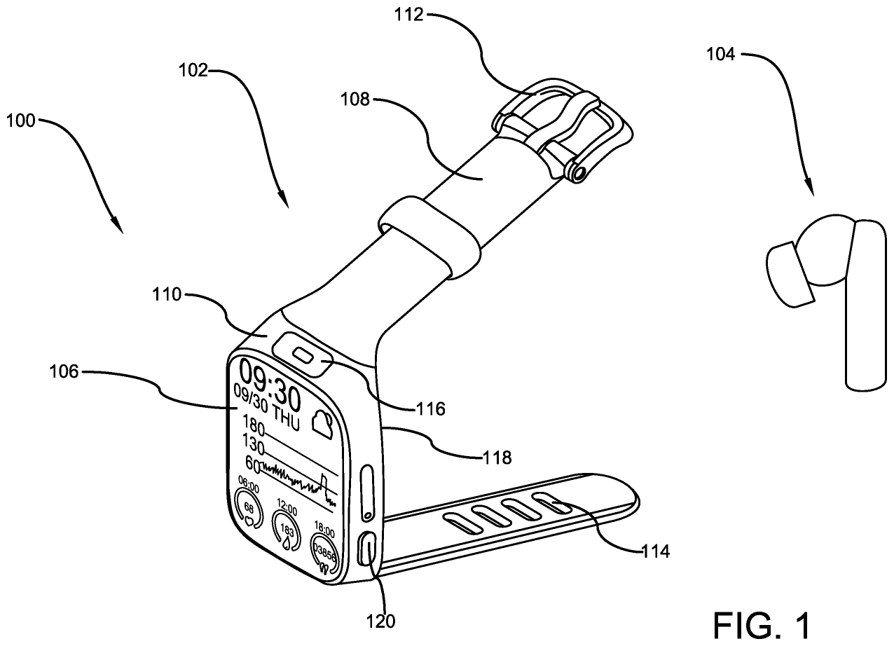

Referring initially to the drawings, FIG. 1 illustrates a perspective view of one potential embodiment of an emotional imbalance detecting smartwatch system of the present invention in accordance with the disclosed architecture. The emotional imbalance detecting smartwatch system 100 of the present invention is designed to help a wearer of the watch 102 cope with negative emotions and emotional imbalance. The system 100 includes a smartwatch 102 and an earbud 104, the earbud 104 is worn in an ear by a wearer of the watch 102. The earbud 104 is wirelessly coupled with the watch 102 such that soothing and relaxing pre-recorded messages and audio are played into the wearer's ear during a negative-emotion event. The wristwatch 102 works such as any conventional smartwatch for detecting vital signs such as heart rate, blood pressure, number of steps, and more and also detects emotions and mood changes of the wearer, thereby detecting negative emotions such as stress and anxiety.

The smartwatch 102 may include a display 106 configured to display vital signs, date, and time information to a wearer of the watch 102. The display 106 may be a touch display and may include one or more light emitting diodes (LED), a two-dimensional LED screen, a two-dimensional liquid crystal display (LCD), and more. The display 106 is configured to receive input from a user such as for configuration of the watch 102. The display 106 is configured to include touch-based, optical, or proximity sensors for detecting information from the user and/or environment.

A strap or band 108 is attached to the housing 110 of the display device 106 and is configured to securely hold the watch 102 around wrist of a wearer. The band 108 has a plurality of holes 114 for receiving a buckle or latch 112 to secure the watch 102 and further for adjusting the size of the loop formed by the band 108. The housing 110 is configured to be positioned against the wearer's body and includes a plurality of sensors for measuring vital signs and emotional state of the wearer as described later in the disclosure. The housing 110 provides a sealed and waterproof casing for all the sensor modules and display of the watch 102.

In one embodiment of the present invention, the housing 110 may include a removeable plate that may be attached utilizing miniature bolts/screws that are sealed utilizing one or more seals. This assists in replacing internal components, such as the battery, buttons, memory, display, and more, as needed by users.

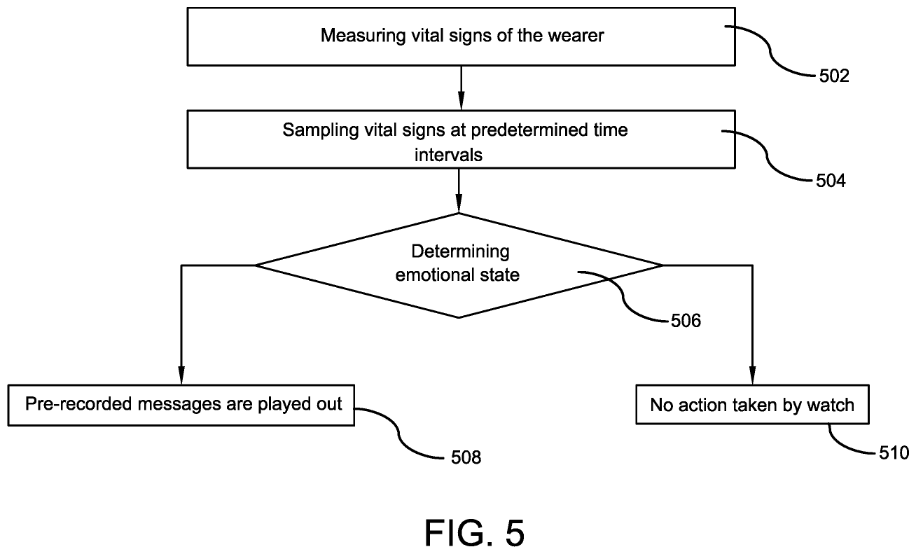

The watch 102 has at least one electrode 116 configured to measure electrical activity of the heart of the wearer of the watch 102. The electrodes 116 helps in checking atrial fibrillation, heart rate variability (HRV), and otherwise analyze the electrical activity of the user wearing the smartwatch 102. An electrodermal activity (EDA) sensor 118 disposed on the housing 110 is used for detecting changes in electrical activity of the skin of the wearer of the watch 102. The smartwatch 102 is configured to detect an emotional state and negative energy level based on the values detected by at least electrodes 116, 118 as described in FIG. 5.

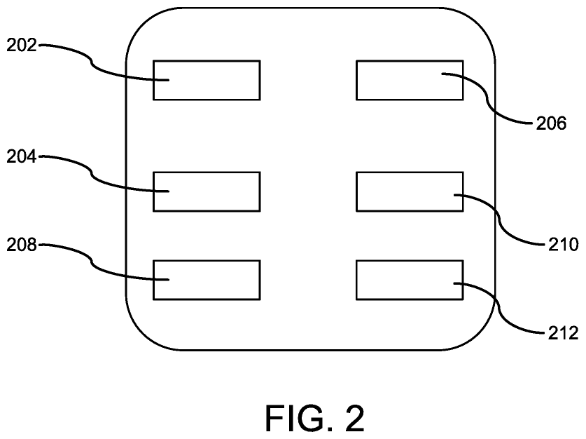

The smartwatch 102 is configured to send an alert message to a remote device when an SOS or help button 120 positioned on the housing 110 is pressed. The help button 120 is manually pressed by a wearer or any other user for requesting and/or obtaining assistance in coping with emotions and feelings. The earbud 104 is wirelessly coupled with the watch 102 and is configured to automatically play a soothing sound stored internally in the smartwatch 102 (as described in FIG. 2) when stress, negative thoughts, and/or emotions are detected by the watch 102.

Both the watch 102 and the earbud 104 may be wirelessly charged using a wireless charger. The watch 102 may have charging pins that are used for receiving a charging cord to recharge the internal battery of the watch 102. The charging pins provide a connection of the internal battery with the electrical power.

The display 106 of the watch 102 is also configured to provide indications indicating the battery charge, estimated battery life remaining, smartwatch status, earbud connection status, wearer status, alerts, and so forth. The wearer status may indicate emotional and mental health of the wearer of the watch and is indicated in a plurality of colors. For example, green may indicate good emotional and mental condition, yellow may indicate that wearer is in stress, and the red may indicate that the wearer needs help/assistance/treatment.

Both the smartwatch 102 and the earbud 104 may have any number of electrical configurations, shapes, and colors and may include various circuitry, connections, and other components. All or portions of the components shown and described with regard to the smartwatch 102 and the earbud 104 may be included in each of the applicable embodiments thereof. Although not specifically shown, the components of the system 100 may be connected or communicate utilizing any number of wires, traces, buses, interfaces, pins, ports, connectors, boards, receptacles, chip sets, transceivers, transmitters, receivers, or so forth.

FIG. 2 illustrates a rear perspective view of the housing showing internal components of the watch 102 in accordance with the disclosed architecture. The present embodiment is configured to illustrate the internal electronic components of the watch 102 of the present invention. The components are used for an effective functioning of the watch 102. The housing 110 of the watch 102 includes a memory 202 for storing vital signs captured by the electrodes and sensors. The memory 202 also includes machine-readable instructions that are executed by the processor 204 for calculating the stress and negative energy based on detected vital signs. The memory 202 stores a plurality of motivational, soothing, and positive messages that are automatically played out in the earbud 104 to help calm down the wearer of the watch 102.

An integrated battery 206 is housed inside the housing 110 and provides required electrical power to the watch 102. The battery 206 is preferably a lithium-ion battery but can also be any button battery, nickel battery, fuel cell battery, solar battery, and the like. For recharging the battery 206, charging pins 208 are disposed which function as a connector for charging the battery 206. The charging pins 208 are also configured to increase or decrease the input voltage level to adjust to the desired voltage level required for recharging the battery 206.

A wireless transceiver 210 is used for enabling the watch 102 to wirelessly connect to the earbud 104 and to transmit an alert message to a remote user device in case of an emergency. The wireless transceiver 210 is in the form an antenna positioned inside the housing 110 and establishes a wireless channel with earbud 104 and a remote device. In some embodiments, the watch 102 may have a sim card that allows the watch 102 to send a SMS or a call to a remote device in case an emergency is detected by the processor 204 of the watch 102.

A location module 212, preferably a Global Positioning System (GPS) device is configured to generate a location signal indicative of a location of the watch 102. In an emergency situation, location of the watch 102 is also provided in the alert message.

The processor 204 may represent any number of microprocessors, digital signal processors (DSP), application-specific integrated circuits (ASIC), field programmable gate arrays (FPGA), or other applicable devices.

The memory 202 is a hardware element, device, or recording media and may represent static or dynamic memory. The memory 202 may include a random-access memory, cache, removable media drive, mass storage, or other construct for storing data, instructions, signals, and/or information.

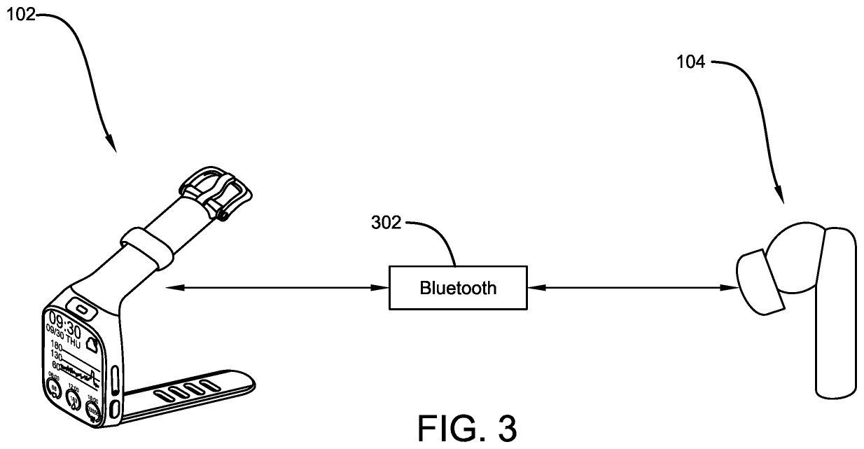

FIG. 3 illustrates a schematic view showing the wireless connection of the smartwatch 102 and the earbud 104 in accordance with the disclosed architecture. The wireless transceiver 210 of the smartwatch 102 establishes a wireless connection 302 with the earbud 104 that allows the pre-recorded messages stored in the memory 202 to be wirelessly transmitted to the earbud 104. The earbud 104 plays out the received messages and helps the wearer in coping with the negative energy, stress, and more. It should be noted that the wireless earbud 104 also includes a wireless transceiver for establishing the Bluetooth channel 302.

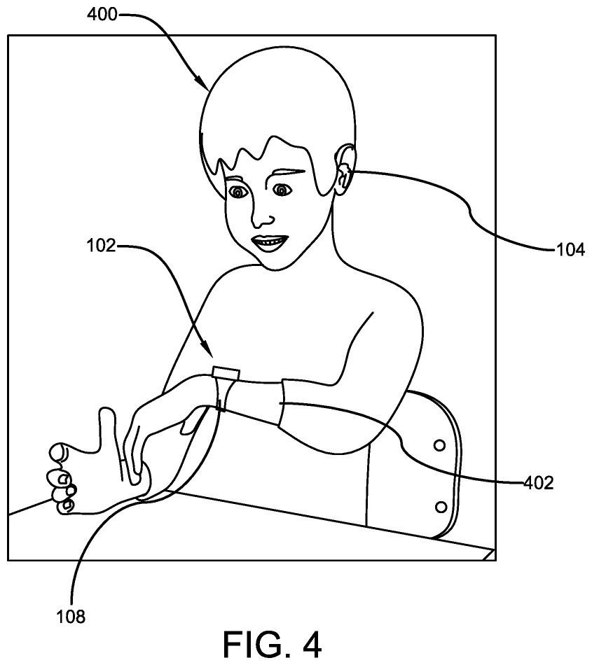

FIG. 4 illustrates a perspective view showing a child using the multipurpose stress relieving watch system 100 for coping with emotions, anxiety, and stress in accordance with the disclosed architecture. As illustrated, the child 400 is wearing the watch 102 with band 108 secured around the wrist 402 such that the electrodes of the watch 102 detects and measures the vital signs of the child 300.

Once the watch 102 detects that the child 400 is stressed, anxious, or having negative emotions, then, automatically, at least one of the pre-recorded messages from the memory of the watch 102 is played out in the earbud 104 to help the child cope with emotions, anxiety, and stress. In some embodiments, the child 400 may select one or more pre-recorded messages using the touch display of the watch 102.

The prerecorded messages can be at least one of counterthoughts, emotions, and encouraging messages and help the wearer identify their emotions and cope with their mood changes.

FIG. 5 illustrates a flow diagram showing the process of determining stress and emotional state of a wearer of the watch 102 in accordance with the disclosed architecture. The processor of the watch 102 includes machine learning algorithms that are configured to process the electrodes or sensors data of the watch 102 to classify and detect an emotional wellbeing of the wearer and accordingly, play out prerecorded messages and/or send an alert signal. For determining the emotional state of the wearer, the electrodes 116, 118 detect and measure various vital signs and electrodermal activities (Step 502). The real time measurements are sampled at predetermined time intervals by the processor to determine HRV and biomarkers (Step 504). Thereafter, the processor, based on the supervised machine learning algorithms, determines an emotional state of the wearer based on the processed sample data of the electrodes (Step 506). If stress, anxiety, or negative mental state is determined, then pre-recorded messages are played out (Step 508). If the emotional state of the wearer is stable, then no action is taken by the watch and the vital signs are displayed on the display as illustrated in FIG. 1 (Step 510).

It should be noted that when the stress, anxiety, or negative mental state is continuously detected for at least a predetermined period of time or the emotional state is found to be on extreme level, then, an alert signal is automatically sent to one or more remote devices of a parent, teacher, or other caregiver to obtain assistance in coping with their emotions and feelings.

The stress relieving and anxiety coping system 100 of the present invention may use various bio signal processing technologies used for emotional state detection, for example, such as skin temperature, blood pressure, blood volume pulses, electroencephalography (EEG), electrocardiography (ECG), electromyography (EMG), and more. The processor may employ both supervised and unsupervised learning and may use profile data of the wearer to accurately determine the emotional state and mood of the wearer. The system 100 can also determine recurring patterns in the emotional instability of the wearer such as time, location, and behavioral pattern of the wearer.

The “electrode” or “sensor” in the present disclosure may refer to a structure used for detecting and/or responding to some type of input from the physical environment, or to one or more of the elements comprised in the whole structure. The electrode may be in the form of a chip that is miniature in size and is electrically coupled to the processor and memory of the watch 102.

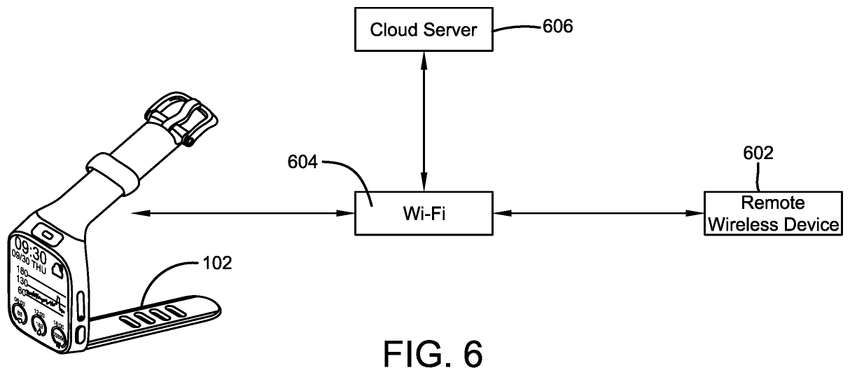

FIG. 6 illustrates a schematic view showing the connection of the watch 102 with a remote wireless device and a cloud server in accordance with the disclosed architecture. The watch 102 is configured to send an alert signal to at least one remote wireless device 602 such as of a parent, teacher, or other caregiver when the SOS or help button 120 of the watch 102 is pressed by wearer of the watch. Also, the watch 102 is configured to automatically send an alert signal or message when the processor of the watch 102 detects a negative or stressed mood for at least a predetermined time to obtain assistance in coping with their emotions and feelings. The alert signal is preferably sent through a Wi-Fi channel 604 and alternatively, Bluetooth, cellphone signals, and/or near-field communication (NFC) radio signals can also be used.

The watch 102 is also configured to connect with a cloud server 606 for storing the measured vital signs of the wearer of the watch 102 and for receiving updated list of pre-recorded messages for storage in an internal memory of the watch 102. The software or operating system of the watch 102 is also configured to receive updates from the cloud server 606 periodically. In one exemplary embodiment, the machine learning classifiers based on demography, gender, age, and profile of the wearer can also be received from the cloud server 606 for enhanced processing and accurate mood and emotion detection of the wearer of the watch.

The wireless device 602 can be a smart phone, a tablet, a laptop, a home computer, and more and can also be utilized to store, display, compile, synchronize, and compile data for the smartwatch 102.

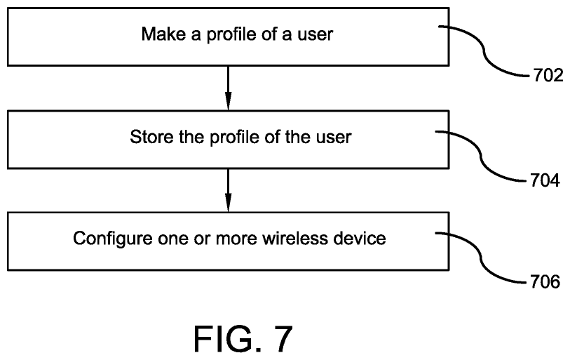

FIG. 7 illustrates a flow diagram depicting a process of associating a user/wearer with the system 100 of the present invention in accordance with the disclosed architecture. Initially, a profile of a user is created in the watch 102 to associate the user with the watch (Step 702). The user is uniquely identified and associated with the watch such that the measurement data is uniquely associated with the user. Profile creation may include gender, demography, age, historical vital signs information, and more.

Once the user profile is created, then the profile is stored in the internal memory of the watch 102 and also on the server 606 (Step 704). Thereafter, one or more wireless devices are configured and coupled with the watch 102 for receiving an alert signal from the watch 102 in case an emergency is detected or SOS button 120 is pressed by the wearer (Step 706).

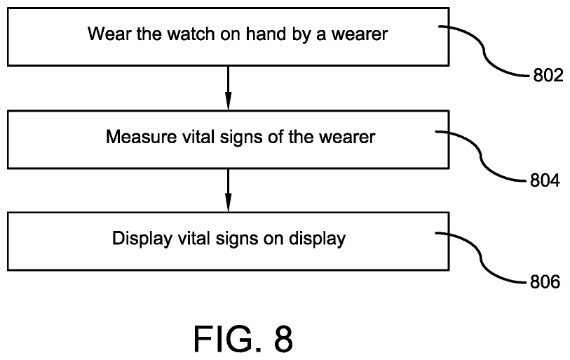

FIG. 8 illustrates a flow diagram depicting a process of measuring and displaying the vital signs and emotional state of a wearer of the smartwatch 102 in accordance with the disclosed architecture. Once the watch 102 is configured for a wearer, the watch 102 is worn and the earbud 104 is plugged in an ear by the wearer (Step 802). The watch 102 can be designed to be worn on a hand, a wrist, around a chest, on a shoulder, and on any other convenient body part as per preferences of the wearer.

Once the watch is positioned on the body of the wearer, the electrodes or sensors start to measure vital signs of the wearer (Step 804). Based on processing of the vital signs, the vital signs along with the emotional state of the wearer is displayed on the display device of the watch 102 (Step 806).

The smartwatch system 100 may be utilized by all age groups, genders, and persons without limitation. The system 100 may be particularly useful for monitoring users that may benefit from or require monitoring for coping with their emotions and feelings.

It should be noted that the smartwatch 102 may include at least one microphone, speaker, and vibrator. The microphone may receive audio commands, content, and sounds from the wearer and the speaker may communicate audio content including indicators, alerts, verbal audio, information, and so forth, to the user. The vibrator can also provide tactile feedback and notices as disclosed with regard to the speakers.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “emotional imbalance detecting smartwatch system”, “smartwatch system”, “multipurpose stress relieving watch system”, and “system” are interchangeable and refer to the multipurpose stress relieving watch system 100 of the present invention.

Notwithstanding the forgoing, the multipurpose stress relieving watch system 100 of the present invention can be of any suitable size and configuration as is known in the art without affecting the overall concept of the invention, provided that it accomplishes the above stated objectives. One of ordinary skill in the art will appreciate that the multipurpose stress relieving watch system 100 as shown in the FIGS. are for illustrative purposes only, and that many other sizes and shapes of the multipurpose stress relieving watch system 100 are well within the scope of the present disclosure. Although the dimensions of the multipurpose stress relieving watch system 100 are important design parameters for user convenience, the multipurpose stress relieving watch system 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A system and device for detecting bio signals of a wearer comprising:
a wearable watch and an earbud worn in an ear by a wearer wherein said watch is wirelessly coupled with said earbud;
wherein said watch having a housing including bio sensors for detecting vital signs selected from a group consisting of a heart rate, a blood pressure, a number of steps, an emotion, and a mood change of the wearer;
wherein detecting said emotion including detecting stress and anxiety levels;
an electrode for measuring electrical activity of a heart of the wearer of said wearable watch wherein said electrical activity of the heart is selected from a group consisting of an atrial fibrillation and a heart rate variability (HRV);
an electrodermal activity (EDA) sensor disposed on an inside surface of said housing for detecting changes in the electrical activity of the skin of the wearer of said wearable watch;
wherein said wearable watch having a plurality of prerecorded messages; and
further wherein at least one of said plurality of prerecorded messages transmitted to said earbud upon detection of said emotion.

2. The system and device for detecting bio signals of a wearer of claim 1, wherein said plurality of prerecorded messages are selected from a group consisting of a soothing message and a relaxing message.

3. The system and device for detecting bio signals of a wearer of claim 2, wherein said emotion is a negative emotion event.

4. The system and device for detecting bio signals of a wearer of claim 3, wherein said soothing message is automatically transmitted upon detection of said negative emotion event.

5. The system and device for detecting bio signals of a wearer of claim 4, wherein said wearable watch having a display for displaying information selected from a group consisting of a vital sign, a date, and a time.

6. The system and device for detecting bio signals of a wearer of claim 5, wherein said display is a touch display having an LED.

7. The system and device for detecting bio signals of a wearer of claim 6, wherein said display having a sensor selected from a group consisting of a touch-based sensor, an optical sensor, and a proximity sensor.

8. The system and device for detecting bio signals of a wearer of claim 7, wherein said wearable watch having a help button including a transceiver for sending an alert message to a remote device for requesting assistance.

9. The system and device for detecting bio signals of a wearer of claim 8, wherein said display having an indicator of wearer status for indicating an emotional status of the wearer.

10. The system and device for detecting bio signals of a wearer of claim 9, wherein said wearer status having a plurality of color displays comprising a first color associated with a first anxiety level, a second color associated with a second anxiety level, and a third color associated with a third anxiety level.

11. The system and device for detecting bio signals of a wearer of claim 10, wherein said wearable watch having a memory for storing said vital signs of said bio sensors.

12. The system and device for detecting bio signals of a wearer of claim 11, wherein said memory having machine-readable instructions executed by a processor for calculating said anxiety levels based upon said vital signs of said bio sensors.

13. The system and device for detecting bio signals of a wearer of claim 12, wherein said memory storing said soothing message and said relaxing message.

14. The system and device for detecting bio signals of a wearer of claim 1 further comprising a location module having a Global Positioning System (GPS) device to generate a location signal indicative of a location of the wearer of said wearable watch.

15. The system and device for detecting bio signals of a wearer of claim 8 further comprising a location module having a Global Positioning System (GPS) device to generate a location signal indicative of a location of the wearer of said wearable watch when said alert message is sent.

16. A system and device for detecting bio signals of a wearer comprising:
a wearable watch and an earbud worn in an ear by a wearer wherein said watch is wirelessly coupled with said earbud;
wherein said watch having a housing including bio sensors for detecting vital signs selected from a group consisting of a heart rate, a blood pressure, a number of steps, an emotion, and a mood change of the wearer;
wherein detecting said emotion including detecting stress and anxiety levels;
an electrode for measuring electrical activity of a heart of the wearer of said wearable watch wherein said electrical activity of the heart is selected from a group consisting of an atrial fibrillation and a heart rate variability (HRV);
an electrodermal activity (EDA) sensor disposed on an inside surface of said housing for detecting changes in the electrical activity of the skin of the wearer of said wearable watch;
wherein said wearable watch having a plurality of prerecorded messages;
wherein at least one of said plurality of prerecorded messages transmitted to said earbud upon detection of said emotion; and
a help button including a transceiver for sending an alert message to a remote device for requesting assistance.

17. The system and device for detecting bio signals of a wearer of claim 16, wherein said plurality of prerecorded messages are selected from a group consisting of a soothing message and a relaxing message.

18. The system and device for detecting bio signals of a wearer of claim 17, wherein said wearable watch having a memory for storing said vital signs of said bio sensors.

19. A system and device for detecting bio signals of a wearer comprising:
a wearable watch and an earbud worn in an ear by a wearer wherein said watch is wirelessly coupled with said earbud;
wherein said watch having a housing including bio sensors for detecting vital signs selected from a group consisting of a heart rate, a blood pressure, a number of steps, an emotion, and a mood change of the wearer;
an electrode for measuring electrical activity of a heart of the wearer of said wearable watch wherein said electrical activity of the heart is selected from a group consisting of an atrial fibrillation and a heart rate variability (HRV);
an electrodermal activity (EDA) sensor disposed on an inside surface of said housing for detecting changes in the electrical activity of the skin of the wearer of said wearable watch;
wherein said wearable watch having a plurality of prerecorded messages;
wherein at least one of said plurality of prerecorded messages transmitted to said earbud upon detection of said emotion;
a help button including a transceiver for sending an alert message to a remote device for requesting assistance; and
a location module having a Global Positioning System (GPS) device to generate a location signal indicative of a location of the wearer of said wearable watch.

20. The system and device for detecting bio signals of a wearer of claim 19, wherein said location signal generated when said alert message is sent.
